(19) United States
(12) Patent Application Publication
(10) Pub. No.: US 2024/0112765 A1
(43) Pub. Date: Apr. 4, 2024

(54) METHOD AND SYSTEM FOR CLINICAL TRIALS MATCHING

(71) Applicant: Codex Genetics Limited, Hong Kong (CN)

(72) Inventors: Shing Hei Tse, Hong Kong (CN); Chi Shing Yu, Hong Kong (CN); Aldrin Kay Yuen Yim, Hong Kong (CN)

(21) Appl. No.: 17/955,700

(22) Filed: Sep. 29, 2022

(51) Int. Cl. G16H 10/20; G06F 40/289; G16B 20/00; G16H 10/60

(52) U.S. Cl. CPC G16H 10/20; G06F 40/289; G16B 20/00; G16H 10/60

(57) ABSTRACT

A computer system for matching clinic trials and patients includes a patient database to store at least one patient profile associated with a patient and a trials database to store at least one trial profile associated with a clinical trial. In response to receiving new information relating to a patient or a clinical trial a feature extraction module determines whether a patient profile or trial profile corresponding to the received information can be found in the database, identifies and extracts features, and activates an update flag. If a corresponding profile is found, the existing profile is updated based on the extracted features. If not found, a new profile is created and stored based on extracted features. An inference module generates a report linking at least one patient with at least one clinical trial based on comparison of extracted features associated with the stored patient profiles and the stored trial profiles.

### TECHNICAL FIELD

The present disclosure relates to identifying potential matches of candidates with clinical trials including matching a single candidate patient or a cohort of candidate patients with clinical trials.

### BACKGROUND

Patients receiving treatment from medical providers accumulate numerous electronic records in a variety of different formats at multiple institutions in receiving treatment for a disease or condition. Typically the information in an electronic health record (EHR) for the patient is in an unstructured format in an inaccessible document format.

For many types of conditions, patients may benefit from directly from participating in medical treatments, drugs or devices under development; with participation of patients in clinical trial collectively essential for ongoing developments in modern evidence-based medicine.

Various inclusion/exclusion criteria may be defined for that medical trial which may determine the suitability of the patient for the trial including the nature and stage of the medical condition, nature and stage of medical treatment, previous treatments etc. Typically such trials may be described on resources such as ClinicalTrials.gov or similar, although many patients/medical professionals lack the ability and/or time to decipher the somewhat abstruse information contained therein.

Unfortunately, there is limited awareness of the existence of such clinical trials and difficulty in accessing and interpreting such information for front line medical professionals treating patients. This is especially marked in Asia in comparison with other regions such as the United States.

If clinicians and researchers are minded to search clinical trials they typically use on domain-specific knowledge and clinical keywords. With a lack of harmonised and standardised set of searching terms, bias and errors may reduce the reliability of any search result. This is especially problematic for serious illnesses such as cancer or other conditions where there may be limited treatment options and limited patients suitable for clinical trials. Ultimately, eligible patients may therefore miss out on possible treatments and clinical trials may lack sufficient candidates to proceed and/or compete for patients with each other.

In an effort to address such deficiencies, rules based or machine learning based approaches to patient matching with clinical trials have been under development. Typically, rules based approaches require patients to answer detailed lengthy questionnaires to provide pre-defined sets of parameters. Such rules based systems may be inaccurate due to errors in the structuring of rules or inability to access and use information contained in patient electronic health records such as phenotype or recent treatment information. Moreover, these questionnaires fail to include any additional information that patients or medical providers may want to add when responding.

Machine based learning approaches do not require such sets of pre-defined parameters in order to make recommendations, yet machine learning approaches are generally not capable of ensuring that certain terms/criteria are to be included, due to statistical nature of trained classifiers. As such, a user may be required to either complete a detailed questionnaire or rely solely on a machine learning prediction for trials recommendations. Most trials recommendations systems in the market therefore rely on a snapshot of a patient profile at the specific point in time when the input parameters are provided.

### SUMMARY

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

In accordance with a first aspect of the present disclosure, there is provided a computer system for matching clinic trials and patients, including a patient database configured to store at least one patient profile associated with a patient a trials database configured to store at least one trial profile associated with a clinical trial, a feature extraction module configured to, in response to receiving new information relating to a patient or a clinical trial: determine whether or not a patient profile or trial profile corresponding to the received information can be found in the database; identify and extract features within the received information; if a corresponding profile is found, update the existing profile based on the extracted features; if a corresponding profile is not found, create and store a new profile based on the extracted features; and activate an update flag; and an inference module configured to, in response to the activation of the update flag, generate a report linking at least one patient with at least one clinical trial based on a comparison of extracted features associated with the stored patient profiles and the stored trial profiles.

The inference module may be configured to generate the report by: performing a pairwise comparison of extracted features associated with the stored patient profiles and extracted features associated with the stored trial profiles, generating a list of extracted feature pairs which is ranked according to the comparison; and selecting the at least one patient and the at least one clinical trial based on at least one entry of the generated list.

The generated report may highlight the features of the at least one entry in the generated list used to select the at least one patient and the at least one clinical trial.

The feature extraction module may include a transformation unit configured to transform text data within the received information to a representative vector.

The feature extraction module may include a genomic data processor configured to identify genomic data in received patient information and compare the identified genomic data with a genome database to extract genome related features.

The feature extraction module may include a biomedical unit configured to recognise extracted features related to one or more cancer entities and introduce additional features identifying the related cancer entities.

The biomedical unit may include a transformer-based entity recognition model and a word embedding classifier model, each trained using a corpus of biomedical data, wherein at least a portion of the corpus is pre-processed to extract noun phrases relating to the one or more cancer entities.

The generated report may include a plurality of clinical trials linked to one patient or a plurality of patients linked to one clinical trial.

In accordance with a second aspect of the present disclosure, there is provided a computer-implemented method for matching clinic trials and patients, including storing, in a patient database, at least one patient profile associated with a patient; storing, in a trials database, at least one trial profile associated with a clinical trial; in response to receiving new information relating to a patient or a clinical trial: identifying and extracting features within the received information; determining whether or not a patient profile or trial profile corresponding to the received information can be found in the database; if a corresponding profile is found, updating the existing profile based on the extracted features; and if a corresponding profile is not found, creating and storing a new profile based on the extracted features; generating a report linking at least one patient with at least one clinical trial based on a comparison of extracted features associated with the stored patient profiles and the stored trial profiles.

Generating the report may include performing a pairwise comparison of extracted features associated with the stored patient profiles and extracted features associated with the stored trial profiles, generating a list of extracted feature pairs which is ranked according to the comparison; and selecting the at least one patient and the at least one clinical trial based on at least one entry of the generated list.

The generated report may highlight the features of the at least one entry in the generated list used to select the at least one patient and the at least one clinical trial with a numerical value representing level of confidence.

Extracting the features may include transforming text data within the received information to a representative vector.

Extracting the features may include identifying genomic data in received patient information and comparing the identified genomic data with a genome database to extract genome related features.

Extracting the features may include recognising extracted features related to one or more cancer entities and introduce additional features identifying the related cancer entities.

Recognising the extracted features related to cancer entities may include activating a transformer-based entity recognition model and a word embedding classifier model, each trained using a corpus of biomedical data, wherein at least a portion of the corpus is pre-processed to extract noun phrases relating to the one or more cancer entities

The generated report may include a plurality of clinical trials linked to one patient or a plurality of patients linked to one clinical trial.

In accordance with a third aspect of the present disclosure, there is provided a computer-readable medium comprising instructions which, when executed by a processor, cause the processor to store, in a patient database, at least one patient profile associated with a patient; store, in a trials database, at least one trial profile associated with a clinical trial; in response to receiving new information relating to a patient or a clinical trial: identify and extract features within the received information; determining whether or not a patient profile or trial profile corresponding to the received information can be found in the database; if a corresponding profile is found, update the existing profile based on the extracted features; and if a corresponding profile is not found, create and store a new profile based on the extracted features; generate a report linking at least one patient with at least one clinical trial based on a comparison of extracted features associated with the stored patient profiles and the stored trial profiles.

Generating the report may include performing a pairwise comparison of extracted features associated with the stored patient profiles and extracted features associated with the stored trial profiles, generating a list of extracted feature pairs which is ranked according to the comparison; and selecting the at least one patient and the at least one clinical trial based on at least one entry of the generated list.

The generated report may highlight the features of the at least one entry in the generated list used to select the at least one patient and the at least one clinical trial with a numerical value representing level of confidence.

Extracting the features may include transforming text data within the received information to a representative vector.

Extracting the features may include identifying genomic data in received patient information and comparing the identified genomic data with a genome database to extract genome related features.

Extracting the features may include recognising extracted features related to one or more cancer entities and introduce additional features identifying the related cancer entities.

Recognising the extracted features related to cancer entities may include activating a transformer-based entity recognition model and a word embedding classifier model, each trained using a corpus of biomedical data, wherein at least a portion of the corpus is pre-processed to extract noun phrases relating to the one or more cancer entities

The generated report may include a plurality of clinical trials linked to one patient or a plurality of patients linked to one clinical trial.

It is an object of the present computer system to address or at least partially ameliorate some of the above problems of the current approaches.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended Figures. Understanding that these Figures depict only exemplary embodiments of the disclosure and are not therefore to be considered to be limiting of its scope, the principles herein are described and explained with additional specificity and detail through the use of the accompanying Figures.

Preferred embodiments of the present disclosure will be explained in further detail below by way of examples and with reference to the accompanying Figures, in which:—

FIG. 1 is a schematic diagram of an embodiment of the present disclosure.

FIG. 2 is a schematic diagram of an example of a feature extraction module according to some embodiments of the present technology.

FIG. 3 illustrates an example method according to some embodiments of the present technology.

FIG. 4 illustrates an example method according to some embodiments of the present technology.

FIG. 5 illustrates an example method according to some embodiments of the present technology.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure.

The disclosed technology addresses the need in the art for an improved computer system for identifying potential matches of candidates with clinical trials.

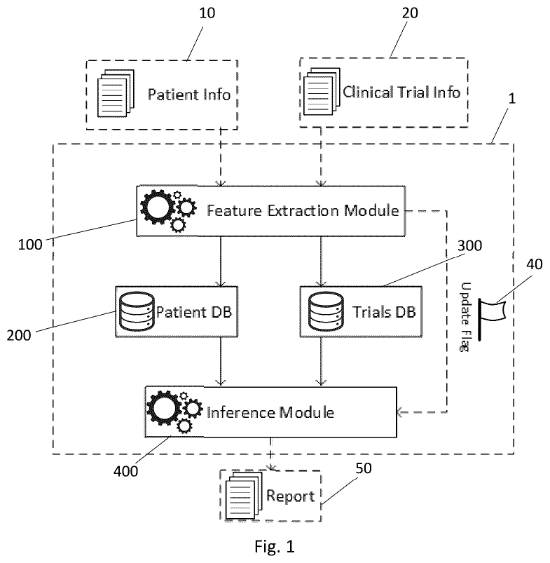

Referring to the drawings, FIG. 1 shows a schematic diagram of a computer system 1 according to an embodiment. The computer system 1 includes a feature extraction module 100, a patient database 200, a trials database 300 and an inference module 400.

The patient database 200 is configured to store at least one patient profile associated with a patient. The patient profile may include one or more phenotypes, biomarkers, demographics etc. relating to a patient. In some examples, the patient profile may be stored in a feature vector form.

The trials database 300 is configured to store at least one trial profile associated with a clinical trial. The trial profile may include one or more phenotypes, biomarkers, demographics etc. relating to a clinical trial, e.g. relating to a desired participant of the clinical trial. In some examples, the trial profile may be stored in a feature vector form.

The feature extraction module 100 is configured to receive information. The feature extraction module 100 may receive patient information and clinical trial information. In response to receiving new information relating to a patient or a clinical trial, the feature extraction module 100 is configured to determine whether or not a patient profile or trial profile corresponding to the received information can be found in the databases. The feature extraction module 100 may perform a database search to discover one or more profiles based on the received new information. In some examples, the new information may be parsed to extract one or more key words for searching the databases.

The feature extraction module 100 is further configured to identify and extract features 30 within the received information. In some examples, the feature extraction module 100 may be configured to search the patient database 200 and/or trials database 300 using the extracted features 30.

If a corresponding profile is found in either of the databases, the feature extraction module 100 is configured to update the existing profile based on the extracted features 30. That is, the feature extraction module 100 may append the received new information to the stored profile. Alternatively, if one or more elements of the received new information correspond to elements of the stored profile, e.g. one or more details have changed, the feature extraction module 100 may replace the corresponding elements of the stored profile with the updated elements of the received new information.

If a corresponding profile is not found, the feature extraction module 100 is configured to create and store a new profile based on the extracted features 30. The new profile may include one or more phenotypes, biomarkers, demographics etc. extracted from the new information. In some examples, the new profile may be stored in a feature vector form.

After updating or creating a profile, the feature extraction module 100 is configured to activate an update flag 40. The update flag 40 may be a binary flag, e.g. a binary variable which is changed from 0 to 1 when “active”.

The inference module 400 is configured to monitor the update flag 40. In response to the activation of the update flag 40, the inference module 400 is configured to generate a report 50 linking at least one patient with at least one clinical trial. In some examples, the generated report 50 may include a plurality of clinical trials linked to one patient. Alternatively, the generated report 50 may include a plurality of patients linked to one clinical trial.

The inference module 400 is configured to generate the report 50 based on a comparison of extracted features 30 associated with the stored patient profiles and the stored trial profiles.

In this way, an improved matching between patient profiles and trial profiles can be provided. A patient user can be provided with a better, more individual recommendation, and an organiser of a clinical trial can be provided with a more useful set of candidates who are more suited to the requirements of the trial. For example, whereas conventional methods are capable only of ranking features based on discrete variables that can be normalized, i.e. clinical features that can be transformed to numbers or a unified format readable by computer, the comparison of extracted features 30 can account for clinical features such as descriptions and medical mentions which vary a lot and are difficult to normalize.

The comparison can be performed immediately upon reception of any new information, whether to generate a new report 50 for a newly received patient or trials profile, or to update an existing report 50. The generated report 50 can be updated at any time, in response to receiving new information, by searching for and updating the corresponding patient and/or trial profiles.

In some examples, the inference module 400 may be configured to perform a pairwise comparison of extracted features 30 associated with the stored patient profiles and extracted features 30 associated with the stored trial profiles. That is, each feature of a patient profile may be compared in a pairwise manner to relevant features of a trial profile, or each feature of a trial profile may be compared in a pairwise manner to relevant features of a patient profile. In some examples, the inference module 400 may be configured to collect all of the extracted features 30 in vector format. In some examples, the inference module 400 may be configured to compute a cosine similarity for each pair of features 30 in vector format.

The inference module 400 may generate a list of extracted feature pairs which is ranked according to the comparison. The linked patient(s) and clinical trial(s) may be selected based on at least one entry of the generated list.

In this way, the inference module 400 can overcome some of the problems with ranking discrete variables or ranking by document level comparisons (e.g. comparing full profiles), such as the inclusion of irrelevant information in the comparison. The inference module 400 can avoid the comparison of features 30 which are dependent on large amounts of textual information that are no directly related to the matching objective. For example, much of the received clinical trial information may include standardised inclusion/exclusion criteria, disclaimers, references and study design, which may appear similar to a document-level comparison system. In comparison, the inference module 400 can consider only the most similar features 30 and dispose of irrelevant features, thereby increasing the specificity of the matching results and increasing the efficiency of the comparison. In addition, the inference module 400 can more easily differentiate between two or more closely matched profiles.

The inference module 400 is not affected by received patient information and clinical trials information which includes a lot of irrelevant description. In addition, the inference module 400 is not affected by patient profiles or trial profiles which are missing certain values or information, as the similarity score of such features 30 is low in the pairwise comparison.

In some examples, the generated report 50 includes a plurality of clinical trials linked to one patient, i.e., the computer system 1 operates to provide trials recommendations to the patient. In some examples, the generated report 50 includes a plurality of patients linked to one clinical trial, i.e., the computer system 1 identifies patients suitable for a clinical trial.

When identifying patients, the inference module 400 may operate in an individual mode or a cohort mode. In a cohort mode, in addition to matching the features 30 of the trial profile, e.g. one or more requirements of the clinical trial, a user may define one or more attributes needed for a desired cohort. For one or more patient profiles matching the features 30 of the trial profile, to recommend a specific cohort, patient features may be semantically compared to the cohort definition (e.g., cancer types, age groups, location, stage of medical conditions such as early stage or late-stage cancer).

In some examples, the generated report 50 may highlight the features 30 of the at least one entry in the generated list used to select the at least one patient and the at least one clinical trial. The highlights may correspond to sentences from the received patient information or clinical trial information. In this way, the generated report 50 can provide evidence to justify the recommendation, instead of operating as a black box.

In some examples, the highlighting may be separated between entity-level features and phrase level features. Entity-level features may include nominal features or features that can be normalised (e.g., age, sex, ethnicity) that can be normalized. Where such features in a patient profile fulfil the requirements of a matched trial profile, a highlight (e.g. with a numerical value representing a level of confidence) may be displayed. A score of either 0 or 1 may be given to these matches to indicate matching as these features can be matched directly. Entity-level features may also include entities/terms that cannot be easily or directly matched (e.g., cancer entities, biomarkers, cancer stage with variable format). For such features, a confidence score may be generated by calculating a cosine similarity score of these features in word vector format with a corpus of reference keywords of respective types. The confidence score can thus validate whether a detected feature is likely to be an entity of the same type.

For phrase level or sentence level features automatically extracted from a patient profile or trial profile, a cosine similarity score can be used to determine the phrase level feature that contributes most to a match. For a trial recommendation, as every patient feature is compared to all criteria in all target trial profiled, then the score highlighted is by default the weighted average of that patient feature to all inclusion/exclusion criteria of that trial profile.

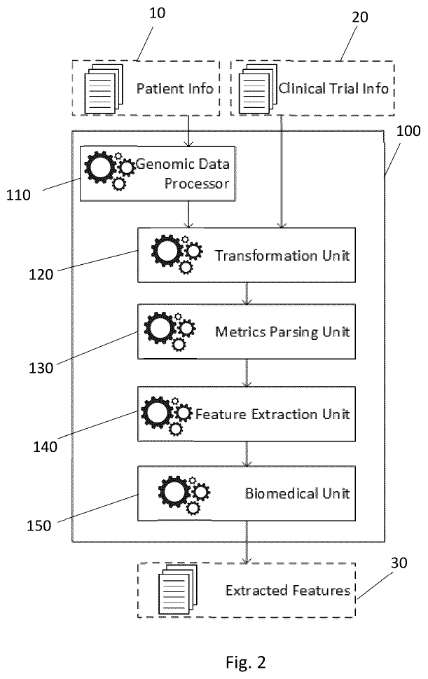

FIG. 2 shows a schematic diagram of the feature extraction module 100 in more detail. The feature extraction module 100 may include a genomic data processor 110, a transformation unit 120, a metrics parsing unit 130, a feature extraction unit 140 and a biomedical unit 150. The feature extraction module 100 may receive patient information and clinical trial information.

The feature extraction module 100 may perform one or more data cleaning operations on the received information. For example, received clinical trial information can be related to public or private clinical trials. For public clinical trials, which may be in a standard format based on the platform or site, a trial parser may automatically extract descriptions, objectives, eligibility criteria and other fields to separate fields for feature extraction.

In some examples, the received patient information may be passed to the genomic data processor 110 and the clinical trial information may be passed to the transformation unit 120.

The genomic data processor 110 may be configured to identify genomic data in received patient information and compare the identified genomic data with a genome database to extract genome related features. For example, the genomic data processor 110 may be configured to identify raw genomic data files e.g. fastq, VCF and BAM formats. The genomic data processor 110 may apply one or more logical filters e.g. using regex and/or rules based on population genetics and clinical significances. The genomic data processor 110 may be configured to identify single nucleotide variations (SNVs), insertions or deletions (indels), amplifications, splice variants and fusions, as well as biomarkers such as tumor mutation burden (TMB) and microsatellite instability (MSI). The filtered data may be compared with a bioinformatics database to extract genome related features. The extracted genome related features may include metrics, predictions scores, biological/clinical/functional impacts etc. In this way, the genomic data processor 110 can provide real-time annotation of variants.

In some examples, the extracted features 30 may be categorised into a plurality of tiers based on clinical significance. For example, a first tier may include variants of strong clinical significance, evidenced by FDA-approved therapy and/or included in professional guidelines or well powered studies with consensus from experts in the field. A second tier may include variants of potential clinical significance, evidenced by FDA-approved investigational therapies and/or multiple small published studies with some consensus, or preclinical trials or a few case reports without consensus. A third tier may include variants of unknown clinical significance, i.e. not observed at a significant allele frequency in general population or specific sub-population databases, with no convincing published evidence of, for example, cancer association. A fourth tier may include benign or likely benign variants, i.e. observed at significant allele frequency in the general population or subpopulation databases, with no existing evidence of cancer association.

The genomic data processor 110 may be configured to generate a clinical interpretation for the most significant tier or tiers. In this way, the genomic data processor 110 can provide a large amount of additional information and related features for the most significant elements of the genomic data. Furthermore, the genomic data processor 110 is agnostic to the specific sequencing technology or data format.

The transformation unit 120 may be configured to transform text data within the received information to a representative vector. For example, the transformation unit 120 may include a transformer model e.g. word2vec.

The metrics parsing unit 130 may be configured to extract one or more terms related to clinical metrics. For example, the metrics parsing unit 130 may use a logical and context-free grammar to parse the received information. The extracted terms may be normalised into units and numerical values.

The feature extraction unit 140 may be implemented as a machine learning model configured to extract features 30 from the received information. For example, the feature extraction unit 140 may utilise a transformer based language model. The feature extraction unit 140 may be configured to extract a patient phenotype, patient demographics, clinical metrics (e.g. age, bloodtype, cancer type etc.), biomarkers, biomedical entities, as well as any descriptions of trials and related phrases. In some examples, the feature extraction unit 140 may be trained using biomedical data from various sources, e.g. MIMIC-III dataset, PubMed articles, publicly available clinical notes etc. In some examples, the feature extraction unit 140 may be configured to separate nominal/ordinal/numerical features from descriptive/sentence level features. In some examples, the feature extraction unit 140 may be implemented as a first model for patient features extraction and a second model for clinical trials feature extraction.

The biomedical unit 150 may be configured to recognise extracted features 30 related to one or more cancer entities and introduce additional features identifying the related cancer entities.

In some examples, the biomedical unit 150 may include a transformer-based entity recognition model and a word embedding classifier model. The entity recognition model may be configured to identify entities in all extracted features 30 and determine if each entity is related to a cancer term. The classifier model may be configured to associate each cancer-related entity with a set of the most common types of cancer, e.g. the top 10 most common types of cancer.

Each of the transformer-based entity recognition model and the word embedding classifier model may be trained using a corpus of biomedical data. In some examples, at least a portion of the corpus may be pre-processed to extract noun phrases relating to the one or more cancer entities. For example, a context-specific resource such as Oncotree or Human Disease Ontology may be used to find reference words related to one or more classes, i.e. specific cancer types. The training corpus may be pre-processed to extract noun phrases containing similar subwords to the reference words.

Further training and evaluation of the biomedical unit 150 may be performed using public biomedical data e.g. PubMed or MIMIC-III Clinical Database.

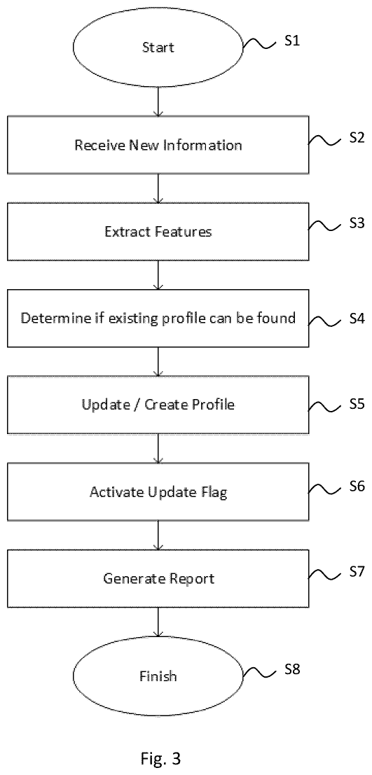

FIG. 3 illustrates an example process flow according to an embodiment. The process flow may be performed by the inference module 400 to find semantically similar features among patients' and clinical trials' features.

At step A1, the inference module 400 may check whether the user wants to perform patient recruitment or trial recommendation. For patient recruitment, the inference module 400 may identify a plurality of patients linked to one clinical trial. For a trial recommendation, the inference module 400 may identify a plurality of clinical trials linked to one patient or cohort of patients.

At step A2, the inference module 400 may check whether the user wants to find trials related to a particular cancer type. If yes, then at step A2-1 the module may identify a plurality of patient profiles and/or trial profiles that are associated a cancer. For example, the inference module 400 may be configured to identify the top 10 most common types of cancer. In some examples, at step A2-2 the inference module 400 may tag the relevant features of the identified profiles with the specific cancer type. In this way, the ranked features produced by the inference module 400 can associated with specific cancer types for later reference.

At step A3, the inference module 400 may collect all of the patient profiles and/or trial profiles, and collect all of the features 30 from the plurality of profiles in vector format. In some examples, for patient recruitment, a particular trial feature set may be collected with all of the patient profile feature sets. If the task is trial recommendation, a particular patient feature set or cohort of patient feature set may be collected with all of the trial profile feature sets.

At step A4, the inference module 400 may be configured to perform a pairwise comparison of features 30. For example, the inference module 400 may include a semantic engine configured to extract similar clinical features by performing pairwise comparison for each patient and trial feature pair. In some examples, the semantic engine may be implemented as a trained machine learning model.

After all pairs have been compared, at step A5 the inference module 400 may be configured to rank the most similar pair of features from the feature space. The inference module 400 may be configured to output an ordered list of features pair depending on the task. For example, for patient recruitment the inference module 400 may output a list of patient profiles linked to one trial profile, and for a trial recommendation the inference module 400 may output a list of trial profiles linked to one patient profile or profile corresponding to a defined cohort group.

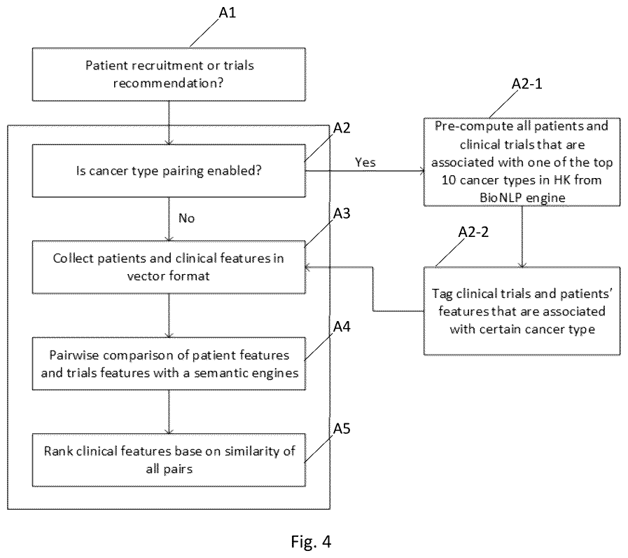

FIG. 4 illustrates an example process flow according to an embodiment. The process flow may be performed by the inference module 400. In this example, at step B1 the inference module 400 determines that the user wants to perform a trials recommendation. The inference module 400 may perform a trials recommendation based on a specific patient profile. The inference module 400 may be configured to extract the patient's attributes or features from the patient profile. In some examples, the features 30 may be extracted in vector format. A plurality of trial profiles may be collected as inputs.

At step B2, the inference module 400 may be configured to filter out one or more trial profiles where the patient fails to be eligible. That is, the inference module 400 may identify one or more trial profiles having mandatory requirements which do not match with the features of the patient profile.

In some examples, soft requirements are also set to minimise the chance of missed matching. At step B3, the inference module 400 may identify any soft requirements and collect trial profiles where there is a partial match between the soft requirements and the features of the patient profile.

At step B4, based on the results of the inference module 400, one or more trials are recommended in a report 50. For example, the top k trials after ranking may be output to the user as a report 50.

At step B5, the inference module 400 may highlight one or more criteria from the trials profiles, e.g. features or phrases, which contributed most to the recommendation.

In some examples, in addition to a one-off recommendation, a patient or authorised user may upload or make changes to their patient profile, e.g. by sending new patient information. In addition, an authorised user may update or make changes to a trials profile e.g. by sending new clinical trial information. On receiving such updated information, the computer system 1 can keep track of the changes in a patient profile and/or trials profile and reinitiate a new recommendation based on the changes, by generating a new report 50 in response to the activation of the update flag 40.

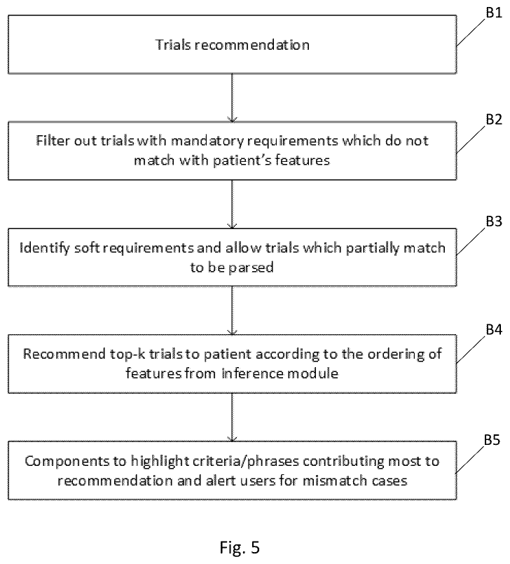

FIG. 5 illustrates a method for matching clinic trials and patients according to an embodiment. The method starts at step S1.

At step S2, the method includes storing at least one patient profile associated with a patient in a patient database 200, and storing at least one trial profile associated with a clinical trial in a trials database 300.

At step S3, the method includes receiving new information relating to a patient or a clinical trial. Steps S4 onwards are performed in response to receiving the new information.

At step S4, the method includes identifying and extracting features 30 within the received information.

In some examples, extracting the features 30 may include transforming text data within the received information to a representative vector.

In some examples, extracting the features 30 may include identifying genomic data in received patient information and comparing the identified genomic data with a genome database to extract genome related features.

In some examples, extracting the features 30 may include recognising extracted features 30 related to one or more cancer entities and introduce additional features identifying the related cancer entities.

In some examples, recognising the extracted features 30 related to cancer entities may include activating a transformer-based entity recognition model and a word embedding classifier model, each trained using a corpus of biomedical data, wherein at least a portion of the corpus may be pre-processed to extract noun phrases relating to the one or more cancer entities

At step S5, the method includes determining whether or not a patient profile or trial profile corresponding to the received information can be found in the database.

At step S6, the method includes updating an existing profile based on the extracted features 30, if a corresponding profile can be found, or creating and storing a new profile based on the extracted features 30 if a corresponding profile is not found.

At step S7, the method includes generating a report 50 linking at least one patient with at least one clinical trial based on a comparison of extracted features 30 associated with the stored patient profiles and the stored trial profiles.

In some examples, generating the report 50 may include performing a pairwise comparison of extracted features 30 associated with the stored patient profiles and extracted features 30 associated with the stored trial profiles, and generating a list of extracted feature pairs which is ranked according to the comparison. At least one patient and at least one clinical trial may be selected based on at least one entry of the generated list.

In some examples, the generated report 50 may highlight the features of the at least one entry in the generated list used to select the at least one patient and the at least one clinical trial.

In some examples, the generated report 50 may include a plurality of clinical trials linked to one patient. Alternatively, the generated report 50 may include a plurality of patients linked to one clinical trial.

The method finishes at step S8.

The above embodiments are described by way of example only. Many variations are possible without departing from the scope of the disclosure as defined in the appended claims.

For clarity of explanation, in some instances the present technology may be presented as including individual functional blocks including functional blocks comprising devices, device components, steps or routines in a method embodied in software, or combinations of hardware and software.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, Universal Serial Bus (USB) devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

1. A computer system for matching clinic trials and patients, comprising:
a patient database configured to store at least one patient profile associated with a patient;
a trials database configured to store at least one trial profile associated with a clinical trial;
a feature extraction module configured to, in response to receiving new information relating to a patient or a clinical trial:
determine whether or not a patient profile or trial profile corresponding to the received information can be found in the respective database;
identify and extract features within the received information;
if a corresponding profile is found, update the existing profile based on the extracted features;
if a corresponding profile is not found, create and store a new profile based on the extracted features; and
activate an update flag; and
an inference module configured to, in response to the activation of the update flag, generate a report linking at least one patient with at least one clinical trial based on a comparison of extracted features associated with the stored patient profiles and the stored trial profiles.

2. The computer system of claim 1, wherein the inference module is configured to generate the report by:
performing a pairwise comparison of extracted features associated with the stored patient profiles and extracted features associated with the stored trial profiles,
generating a list of extracted feature pairs which is ranked according to the comparison; and
selecting the at least one patient and the at least one clinical trial based on at least one entry of the generated list.

3. The computer system of claim 2, wherein the generated report highlights the features of the at least one entry in the generated list used to select the at least one patient and the at least one clinical trial.

4. The computer system of claim 1, wherein the feature extraction module includes a transformation unit configured to transform text data within the received information to a representative vector.

5. The computer system of claim 1, wherein the feature extraction module includes a genomic data processor configured to identify genomic data in received patient information and compare the identified genomic data with a genome database to extract genome related features.

6. The computer system of claim 1, wherein the feature extraction module includes a biomedical unit configured to recognise extracted features related to one or more cancer entities and introduce additional features identifying the related cancer entities.

7. The computer system of claim 6, wherein the biomedical unit comprises a transformer-based entity recognition model and a word embedding classifier model, each trained using a corpus of biomedical data, wherein at least a portion of the corpus is pre-processed to extract noun phrases relating to the one or more cancer entities.

8. The computer system of claim 1, wherein the generated report includes a plurality of clinical trials linked to one patient or a plurality of patients linked to one clinical trial.

9. A computer-implemented method for matching clinic trials and patients, comprising:
storing, in a patient database, at least one patient profile associated with a patient;
storing, in a trials database, at least one trial profile associated with a clinical trial;
in response to receiving new information relating to a patient or a clinical trial:
identifying and extracting features within the received information;
determining whether or not a patient profile or trial profile corresponding to the received information can be found in the respective database;
if a corresponding profile is found, updating the existing profile based on the extracted features; and
if a corresponding profile is not found, creating and storing a new profile based on the extracted features;
generating a report linking at least one patient with at least one clinical trial based on a comparison of extracted features associated with the stored patient profiles and the stored trial profiles.

10. The computer-implemented method of claim 9, wherein generating the report comprises:
performing a pairwise comparison of extracted features associated with the stored patient profiles and extracted features associated with the stored trial profiles,
generating a list of extracted feature pairs which is ranked according to the comparison; and
selecting the at least one patient and the at least one clinical trial based on at least one entry of the generated list.

11. The computer-implemented method of claim 10, wherein the generated report highlights the features of the at least one entry in the generated list used to select the at least one patient and the at least one clinical trial with a numerical value representing level of confidence.

12. The computer-implemented method of claim 9, extracting the features includes transforming text data within the received information to a representative vector.

13. The computer-implemented method of claim 9, extracting the features includes identifying genomic data in received patient information and comparing the identified genomic data with a genome database to extract genome related features.

14. The computer-implemented method of claim 9, wherein extracting the features includes recognising extracted features related to one or more cancer entities and introduce additional features identifying the related cancer entities.

15. The computer-implemented method of claim 14, wherein recognising the extracted features related to cancer entities includes activating a transformer-based entity recognition model and a word embedding classifier model, each trained using a corpus of biomedical data, wherein at least a portion of the corpus is pre-processed to extract noun phrases relating to the one or more cancer entities

16. The computer-implemented method of claim 9, wherein the generated report includes a plurality of clinical trials linked to one patient or a plurality of patients linked to one clinical trial.

17. A computer-readable medium comprising instructions which, when executed by a processor, cause the processor to perform the method of claim 9.
